(19) United States
(12) Patent Application Publication
(10) Pub. No.: US 2026/0090905 A1
(43) Pub. Date: Apr. 2, 2026

(54) BILATERAL TONGUE BASE REPOSITIONER

(71) Applicant: John Summer, Portland, OR (US)

(72) Inventor: John Summer, Portland, OR (US)

(21) Appl. No.: 18/904,077

(22) Filed: Oct. 2, 2024

(51) Int. Cl. A61F 5/56

(52) U.S. Cl. CPC A61F 5/566

(57) ABSTRACT

A bilateral tongue base repositioner holds the lateral borders of the tongue base inferiorly and anteriorly by means of lateral tongue base contacting pads located at the posterior ends of a wire framework that rotates around and slides through slots at the posterior ends of the buccal walls of an upper oral appliance. The contour of the lateral tongue base contacting pads is flat transversely, with an inferior and anterior facing concavity. The inferiorly and anteriorly facing surfaces of the lateral tongue base contacting pads also have an intricately textured tongue gripping surface provided by transversely oriented grooves with anteriorly slanted walls that grip the tissue on top of the sides of the tongue base to prevent it from slipping posteriorly into the pharynx. The wire framework comprises two antero-posteriorly oriented arms that are connected posteriorly by a transverse member. Each arm has an anterior end that is bent sharply inward (medially) to engage an apertured positioning plate attached to the buccal wall of the upper oral appliance. The anterior ends of the arms can be forced laterally out of one set of apertures and shifted into a different set of apertures to adjust the position of the lateral tongue base contacting pads.

### CROSS-REFERENCE TO RELATED APPLICATIONS

Application No. 63/629,217 from Provisional filed Oct. 1, 2023

### PRIOR ART

In U.S. Pat. Nos. 6,766,802, 6,766,802, and 6,766,802; Keropian teaches a posterior tongue restrainer comprising a centrally located tongue base contacting pad which controls the tongue base by depressing it, and it is attached to an upper oral appliance by a transpalatal bar. However, Keropian's posterior tongue restrainer and the transpalatal bar supporting it are too rigid to accommodate the natural movement of the tongue base during swallowing, which makes it uncomfortable; and Keropian's tongue base contacting pad only contacts the tongue base in its medial half, where the gag reflexes are located; thus, its posterior reach is limited. Also, Keropian's tongue base contacting pad employs a smooth tissue contacting surface, which allows the tongue base to slide easily beneath it; therefore, the inferiorly directed pressure can force the tongue base down and back into the area of obstruction, exacerbating the same problem it is trying to solve.

U.S. Pat. No. 10,383,758-B1 teaches a transpalatal strap that contacts both the medial and lateral aspects of the tongue base. It exerts some control on the position of the tongue base by pushing it downward, which addresses some obstructions that occur superiorly in the nasopharynx; but it can exacerbate obstructions down lower in the hypopharynx, like Keropian's posterior tongue restrainer. Also like in Keropian's posterior tongue restrainer, the contact in the medial half of the tongue base limits the distance in which the hardware can extend posteriorly.

U.S. Pat. Nos. 9,808,371 B2 and 9,801,755 B2 to Summer teach a tongue depressor that reaches posteriorly from the posterior end of an oral appliance. Although not shown explicitly, it could reach far enough posteriorly to contact the tongue base; where it could control the position of the tongue base by pushing it inferiorly; but the tongue contacting portions still contact the medial half of the tongue.

Thus, there is a need for a device that can be added to an upper oral base appliance to control the position of the tongue base during sleep by its lateral borders where there are few gag reflexes, and it should be flexible enough to accommodate the powerful contraction of the tongue base muscles during swallowing, when breathing stops anyway.

### BACKGROUND OF THE INVENTION

Obstructive sleep apnea is a process of repeatedly choking on the tongue base. The loose soft tissues surrounding the tongue base may function like a gasket to complete the seal between the tongue base and the pharyngeal wall, and they often get blamed for causing the condition, but the tongue base functions as the stopper; therefore, effective treatment for obstructive sleep apnea requires controlling the position of the tongue base during sleep. However, the medial portion of the tongue base is untouchable due to the density of gag reflexes there.

### SUMMARY OF THE INVENTION

The current invention repositions the tongue base by controlling the positions of two lateral tongue base contacting pads at the posterior ends of an antero-posteriorly elongated wire framework that slides through and rotates around slots projecting from the posterior ends of the buccal walls of an upper oral appliance. The lateral tongue base contacting pads avoid any contact with the medial half of the tongue base, where the gag reflexes are located, and they taper further away from the midline and the medial half of the tongue base as they extend further posteriorly. From a side view, the lateral tongue base contacting pads have inferiorly and anteriorly facing concave curves that fit the superior surface of the lateral borders of the tongue base, and they contain transverse grooves, preferably anteriorly slanted, to prevent the tissues of the tongue base from sliding posteriorly beneath them. The wire framework comprises two antero-posteriorly elongated arms that are connected posteriorly at a location beyond the posterior end of the oral appliance. The arms have posterior ends fixed to lateral tongue base controlling pads, middle portions that slideably and rotatably engage slots in brackets projecting from the posterior end portions of the upper oral appliance, and anterior ends that comprise short sharply bent segments that engage apertures in apertured positioning plates bonded to the buccal walls of the oral appliance. By moving the anterior ends of the arms between different sets of apertures in the apertured positioning plates, the locations of the lateral tongue base contacting pads can be controlled.

### BRIEF DESCRIPTION OF DRAWINGS

FIG. 1 shows a top view of the first described embodiment.

FIG. 2 shows a side view of the described embodiment in a position to facilitate insertion.

FIG. 3 shows a rear view of the described embodiment.

FIG. 4 shows the side view of FIG. 2 after shifting the wire framework posteriorly.

FIG. 5 shows the side view of FIG. 4 after further rotating the wire framework.

### DETAILED DESCRIPTION OF THE INVENTION

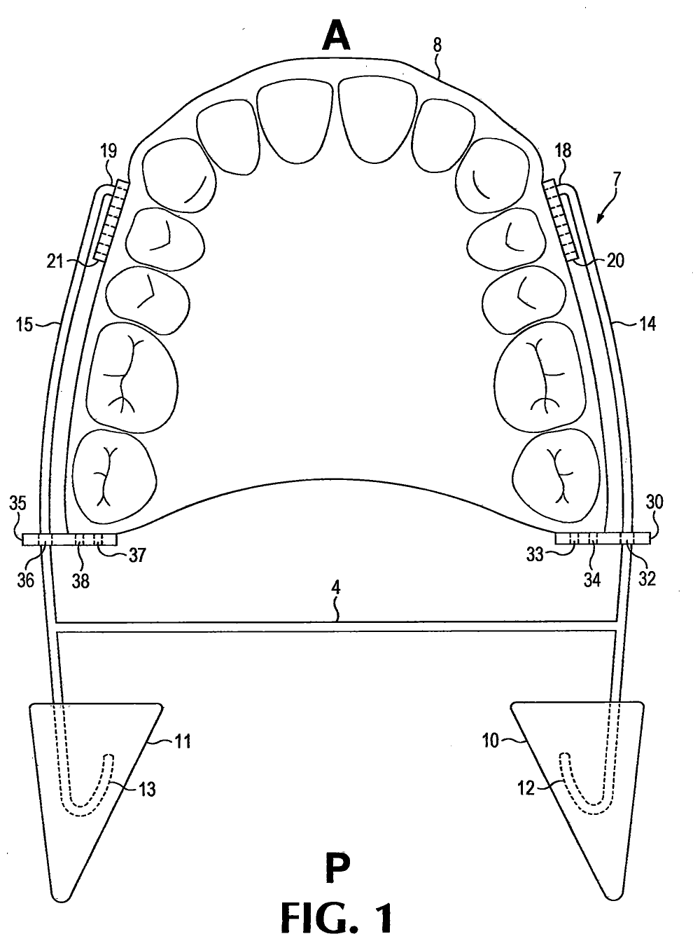

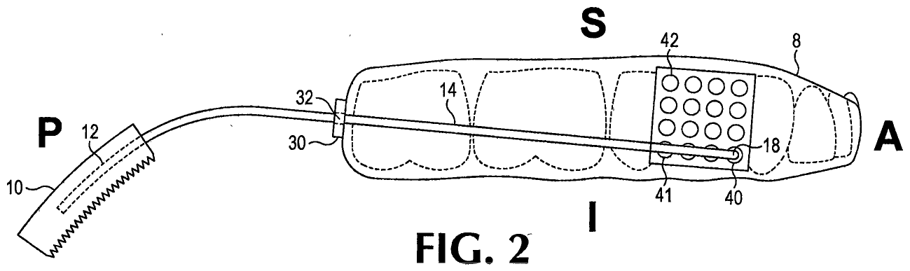

In FIG. 1, upper oral appliance 8 is a removeable resinous shell custom made to fit closely over the user's upper teeth. It has an anterior end, signified by a large black A and a posterior end signified by a large black P. The appliance also has a superior side, signified by a large black S, and an inferior side, signified by a large black I in FIG. 2. It also has right and left buccal walls, its outer walls.

These oral appliances are normally made of sheet acrylic and sometimes with orthodontic acrylic. However, a variety of other materials and appliance designs could be employed to hold onto the upper teeth and also to engage the wire framework as described below. For example, various mandibular advancement appliances could be used to support and engage the wire framework.

FIG. 2 shows the illustrated embodiment from one side view. The other side is identical, but not shown.

As seen in FIGS. 1-3; wire framework 7 is comprised of two antero-posteriorly elongated wires 14 and 15 of diameter about 0.030″-0.050″. The anterior portions of wire framework 7 are located adjacent to the buccal (outer) walls of the oral appliance 8. The posterior portions of wire framework 7 extend posteriorly past the posterior ends of oral appliance 8.

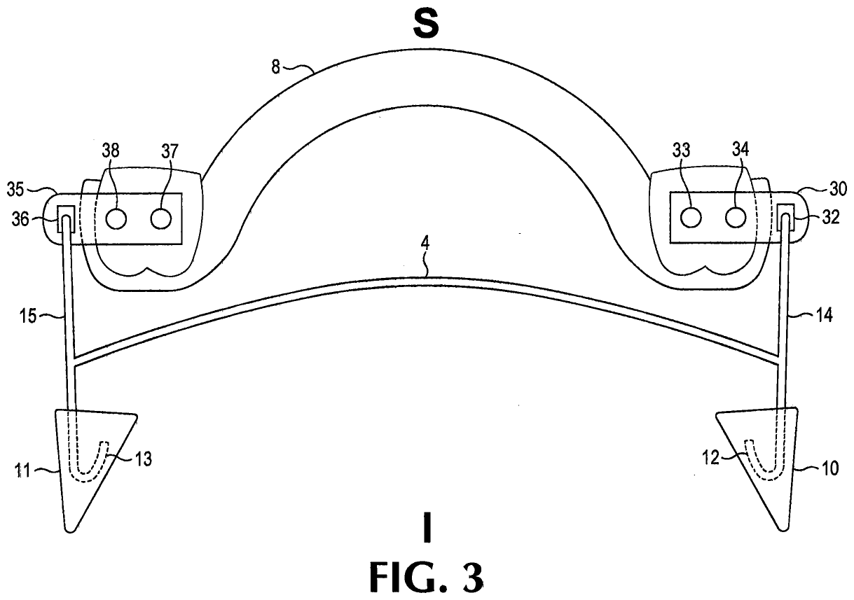

Wires 14 and 15 are connected posteriorly at a location beyond the posterior end of upper oral appliance 8 by transverse connector 4. Transverse connector 4 is illustrated as a simple wire, but it could also comprise a length of sheet metal, such as a 0.2″ wide strip of 0.020″ thick stainless steel. The strip or wire is easily soldered or welded to wires 14 and 15. Transverse connector 4 is bent into a downward facing concave curve, generally parallel with the curve of the soft palate just above it, as can be seen in FIG. 3. Such a curve is not necessary for the basic operation of the apparatus, but it gives the wire framework 7 a resiliency that keeps its anterior ends seated in the apertured positioning plate, as described below.

The posterior end portions 12 and 13 of wires 14 and 15 respectively form loops that enable them to be fixed to the lateral tongue base contacting pads 10 and 11 respectively by embedding them in acrylic. Secure attachment of the lateral tongue base contacting pads 10 and 11 to the posterior ends 12 and 13 respectively is necessary, because any dislodging of a part could be dangerous at a location over an open airway. The lateral tongue base contacting pads 10 and 11 make no contact with the medial half of the tongue base, the portion along its midline, where the gag reflexes are located.

In addition, as the lateral tongue base contacting pads 10 and 11 extend further posteriorly, they taper further away from the midline of the tongue base and thereby contact a progressively smaller portion of its lateral borders until they contact only its lateral edges. In FIG. 1, the lateral tongue base contacting pads 10 and 11 form triangular shapes with their rounded apices located postero-laterally. However, the triangular shape is not necessary. For example, the lateral tongue base contacting pads could be just narrow strips contacting only the lateral edges of the lateral borders of the tongue base.

As shown in FIG. 2, the lateral tongue base contacting pad 10 is anatomically contoured antero-posteriorly, with a slight downward facing concavity, generally parallel with the contour of the tongue base in that region. While the superior and posteriorly facing surfaces of the lateral tongue base contacting pads are smooth, their inferior and anteriorly facing surfaces are textured to grip the tissue on top of the lateral borders of the tongue base to prevent it from slipping down and back into the pharynx. The texture is preferably provided by transverse grooves that have anteriorly projecting walls, as seen in FIGS. 2 and 3, to prevent the tongue base from sliding posteriorly beneath them.

At the posterior ends of oral appliance 8, brackets 30 and 35 are rectangular panels comprised of sheet metal of thickness 0.015-0.025″. Each bracket is about ⅜″-½″ long and ¼″-⅛″ tall. Bracket 30 contains slot 32 and retentive apertures 33 and 34; while bracket 35 contains slot 36 and retentive apertures 37 and 38. While the illustrated embodiment shows two large retentive apertures on each bracket, multiple smaller retentive apertures or even just notches in the sides of the bracket could also be used for retention. The retentive apertures work by providing a space for penetration of the resin of which the oral appliance is made, but apertures are just one of many possible methods for securing the bonding of the brackets 30 and 35 to the posterior ends of the oral appliance 8. In addition, L-shaped brackets could be used with the retentive leg of the L bonded to the posterior buccal wall of the oral appliance rather than to its posterior end, and the leg of the L which contains the slot that engages the wire arm still projecting buccally from the posterior end portion of the buccal walls, without departing from the spirit of the invention. The slots engage wires 14 and 15 generally in their midportions, but the location at which the wires engage the slots can vary to fit individual anatomy.

The slots 32 and 36 are about 0.035 to 0.045″ tall vertically, tall enough to allow rotation of the 0.030″ diameter wires on a vertical plane within the slots. The wire framework 7 can also be slid anteriorly or posteriorly through slots 32 and 36, and the sliding and rotation can be combined in moving the wire framework between apertures, thereby combining inferior and anterior repositioning of the tongue base in various combinations. Simple slots are used in the illustrated embodiment, but more complex engagement mechanisms, such as a tubing on a hinge, could serve the same function without departing from the spirit of the invention.

The anterior ends 18 and 19 of arms 14 and 15, comprise short (3-4 mm long) sharply bent segments that diverge at about a ninety degree angle from arms 14 and 15; but the exact angle used can vary to fit varied anatomy, such as the angle of the buccal wall in the premolar region. For example, the anterior ends 18 and 19 are oriented perpendicular to the apertured positioning plates 20 and 21, while the orientation of the arms 14 and 15 from which the anterior ends 18 and 19 diverge is oriented parallel to the buccal walls of the premolar region of the oral appliance. Therefore, the angle at which the anterior ends 18 and 19 diverge from the rest of arms 14 and 15 could be slightly more or less than ninety degrees.

The anterior ends 18 and 19 of arms 14 and 15 respectively engage the apertures in the apertured positioning plates 20 and 21 respectively, which are embedded in the buccal walls of upper oral appliance 8. In the illustrated embodiment, the apertured positioning plates 20 and 21 are embedded in the first premolar area of the buccal walls, however they could be embedded as far posteriorly as the molars and as far anteriorly as the canines, without departing from the spirit of the invention.

Apertured positioning plates 20 and 21 in the illustrated embodiment are each square pieces of stainless steel sheet metal that contain 16 apertures, however they could be rectangular or of any shape that fits on and can be bonded to the buccal walls. Such apertured stainless steel sheets are readily available. The shapes and number of apertures could be varied without departing from the spirit of the invention. Each of the apertures in the apertured positioning plates is only about 0.001″-0.002″ larger in diameter than the 0.030″ diameter wires which engage them in order to ensure a secure engagement of the anterior ends of the arms in the apertured positioning plates.

The resilience of the wire framework keeps the anterior ends 18 and 19 fully seated within the apertures in the apertured positioning plates. The anterior ends 18 and 19 can be shifted between apertures by forcing them laterally out from one set of apertures, moving them into a position alongside a different set of apertures, and then releasing them to allow the natural bias from the resilience of the wire framework to seat them into the different set of apertures.

The anterior ends of the arms can be pulled out of one set of apertures by using a simple tool like a tweezer or fingers and then shifted into a different set of apertures while the oral appliance is in the user's mouth. For example, the anterior ends of the arms can be set in the most inferior and anterior apertures during insertion of the device to minimize the contact with the gag reflexes in the lateral borders of the tongue base, then shifted superiorly and/or posteriorly to push the lateral borders of the tongue base into an optimal treatment position. FIG. 2 shows one side of the apparatus in that insertion position.

Alternatively, when the apparatus in not in the user's mouth, the anterior ends of the arms can be forced out of one set of apertures and then shifted into a different set of apertures by squeezing together the posterior ends of arms 14 and 15, which increases the inferiorly facing concavity in the transverse connector 4 and causes arms 14 and 15 to rotate in a horizontal plane around slots 32 and 36. Then wire framework can be repositioned, and then its anterior ends 18 and 19 can be released, enabling the resilience of the wire framework to seat its anterior ends 18 and 19 into the different set of apertures. In the illustrated embodiment, slots 32 and 36 are each about 0.003″-0.005″ wider than the thickness of the arms to allow sufficient horizontal rotation of arms 14 and 15 to force their anterior ends out of the apertures in the apertured positioning plates.

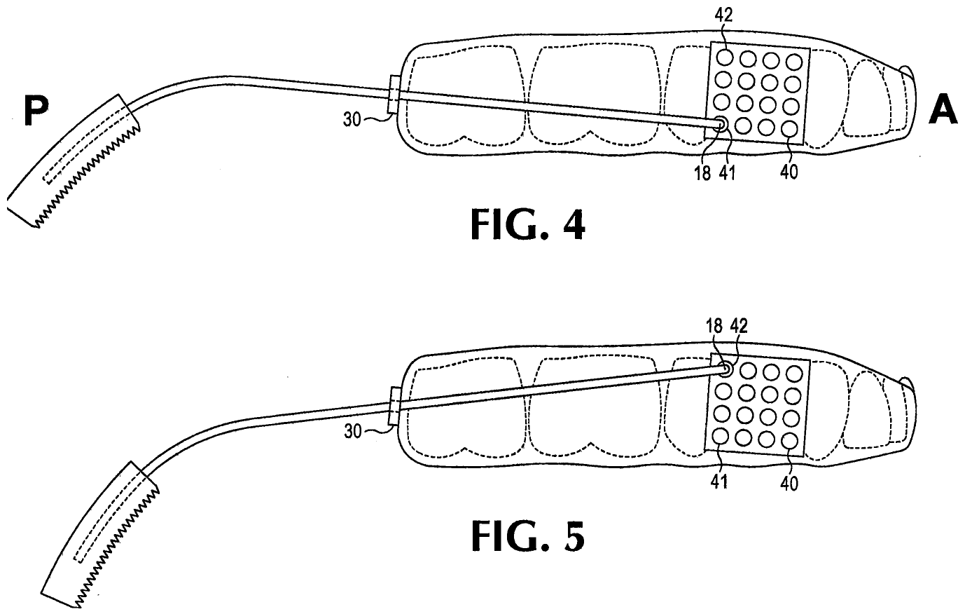

In FIG. 2, the anterior end 18 of arm 14 is engaged in aperture 40, which positions the lateral tongue base contacting pad 10 as superiorly and anteriorly as possible. This position facilitates insertion of the appliance. In FIG. 4, the wire framework has been shifted posteriorly from that insertion position as far as possible by shifting its anterior end 18 (shown) posteriorly into aperture 41. This adjustment positions the lateral tongue base contacting pads further behind the lateral borders of the tongue base to get them in more advantageous positions from where they can more effectively rotate the lateral borders of the tongue base anteriorly and inferiorly. In FIG. 5, the wire framework has been rotated from that advantageous posterior position by shifting its anterior end 18 superiorly from aperture 41 into aperture 42.

Instead of using apertured positioning plates, the device could employ other means of fixing the anterior ends of the arms to the buccal walls of an oral appliance. For example, the anterior ends of the arms could be clamped against a simple non-apertured positioning plate, or two parallel positioning plates can be clamped together by a bolt and nut in order to lock the anterior ends of the arms in the desired position by compressing them between the positioning plates. Alternatively, the anterior ends of the arms could be left unrestrained for insertion of the device and then shifted superiorly into a position where they can be stabilized, such as by locking them on a ledge or even just embedded in acrylic.

1. An apparatus for repositioning a user's tongue base, which has a medial half and two lateral borders; comprises an upper oral appliance, which has anterior and a posterior ends, superior and inferior sides, and right and left buccal walls; and a wire framework that slideably and rotatably engages the posterior end portions of the buccal walls; wherein the wire framework comprises two antero-posteriorly elongated arms that are connected by a transverse connector at a location posteriorly beyond the posterior end of the upper oral appliance; and wherein each wire arm comprises a posterior end that is fixed to a lateral tongue base contacting pad that contacts a lateral border of the tongue base, a middle portion that rotatably and slideably engages a slot projecting from the buccal wall of the posterior end portion of the oral appliance, and an anterior end that comprises a sharply bent segment that engages an apertured positioning plate on the buccal wall of the upper oral appliance.

2. The apparatus of claim 1 wherein the inferiorly and anteriorly facing surfaces of the lateral tongue base contacting pads are anatomically contoured to fit the natural curve of the lateral borders of the tongue base.

3. The apparatus of claim 2 wherein the inferiorly and anteriorly facing surfaces of the lateral tongue base contacting pads are textured to grip the tissues on the lateral borders of the tongue base.

4. The apparatus of claim 3 wherein the texturing of the inferiorly and anteriorly facing surfaces of the lateral tongue base contacting pads comprises transversely aligned grooves that are slanted anteriorly to prevent the tongue base from sliding posteriorly beneath them.

5. The apparatus of claim 1 wherein the anterior ends of the arms comprise sharply bent segments that are held in the apertured positioning plate by the resilience of the wire framework.

6. The apparatus of claim 5 wherein the anterior ends of the arms can be forced laterally out of one set of apertures by squeezing together the posterior ends of the arms, shifted to a location alongside a different set of apertures, and then released into that different set of apertures by releasing the posterior ends of the arms.

7. An apparatus that repositions the tongue base, which has a medial half located between its two lateral borders, inferiorly and anteriorly during sleep by its lateral borders; the apparatus comprising an upper oral appliance, which has anterior and posterior ends and right and left buccal walls; and a wire framework that slideably and rotatably engages the posterior end portions of the buccal walls.

8. The apparatus of claim 7 wherein the wire framework comprises two antero-posteriorly elongated arms connected at a location posterior to the posterior end of the upper oral appliance; and wherein each arm has a posterior end that is fixed to a lateral tongue base contacting pad, a midportion that pivotally and slideably engages a slot at the posterior end portion of the oral appliance, and an anterior end that can be variably positioned on the buccal wall of the oral appliance.

9. The apparatus of claim 8 wherein the variable positioning of each anterior end comprises sharply bent segments that engage apertures in an apertured positioning plate on the buccal wall of the oral appliance.

10. The apparatus of claim 9 wherein the anterior ends of the arms can be forced laterally out of one set of apertures and shifted to a different set of apertures to shift the positions of the lateral tongue base contacting pads.

11. The apparatus of claim 7 wherein the oral appliance also has superior and inferior sides, and wherein the inferiorly and anteriorly facing surfaces of the lateral tongue base contacting pads are textured to grip the tissues on the lateral borders of the tongue base.

12. The apparatus of claim 11 wherein the texturing of the inferior surfaces of the contacting pads comprises transversely aligned grooves to prevent the lateral borders of the tongue base from sliding posteriorly beneath them.

13. A method for keeping the pharyngeal airway open, the method comprising repositioning the lateral borders of the tongue base, which comprise only the lateral fourths of the tongue base, inferiorly and anteriorly during sleep by pushing on them with a wire framework that pivotally and slideably engages the posterior end portions of an upper oral appliance; which has anterior and posterior ends, superior and inferior sides, and right and left buccal walls; to apply force anteriorly and inferiorly on lateral tongue base contacting pads that contact the lateral borders of the tongue base.

14. The method of claim 13 wherein the wire framework comprises two wire arms that pivotally and slideably engage the posterior end portions of the right and left buccal walls.

15. The method of claim 14 wherein the wire arms pivotally and slideably engage slots protruding laterally from the posterior end portions of the right and left buccal walls.

16. The method of claim 13 wherein the lateral tongue base contacting pads have inferiorly and anteriorly facing concave curves.

17. The method of claim 13 wherein the anterior ends of the wire arms comprise sharply bent segments that engage apertures in apertured positioning plates bonded to the buccal walls of the oral appliance.

18. The method of claim 17 wherein the resilience of the wire framework keeps the anterior ends of the wire arms seated in apertures in the apertured positioning plates and the anterior ends of the wire arms can be forced out of one set of apertures in the apertured positioning plates, shifted, and allowed to seat into a different set of apertures.

19. The method of claim 18 wherein the resilience of the wire framework keeps the anterior ends of the wire arms seated in apertures in the apertured positioning plates and the anterior ends of the wire arms can be pulled out of the first set of apertures manually using fingers or a simple tool and allowed to seat into a different set of apertures while the oral appliance is in the user's mouth.

20. The method of claim 17 wherein the resilience of the wire framework keeps the anterior ends of the wire arms seated in apertures in the apertured positioning plates, and the anterior ends of the wire arms can be forced out of one set of apertures in the apertured positioning plates by squeezing together the posterior ends of the wire arms.
